(19) United States
(12) Patent Application Publication
(10) Pub. No.: US 2026/0144773 A1
(43) Pub. Date: May 28, 2026

(54) METHODS OF REDUCING PHYSICAL DEPENDENCE TO NEUROPSYCHIATRIC TREATMENTS

(71) Applicant: Sumitomo Pharma America, Inc., Marlborough, MA (US)

(72) Inventors: Hailong CHENG, San Jose, CA (US); Heather DWORAK, Bloomfield, NJ (US); Colleen SYNAN, Townsend, MA (US)

(73) Assignee: Sumitomo Pharma America, Inc., Marlborough, MA (US)

(21) Appl. No.: 19/119,851

(22) PCT Filed: Oct. 13, 2023
(86) PCT No.: PCT/US2023/076761

§ 371 Date: Apr. 10, 2025

Related U.S. Application Data
(60) Provisional application No. 63/379,364, filed on Oct. 13, 2022.

(51) Int. Cl. A61K 31/381; A61P 25/18

(52) U.S. Cl. CPC A61K 31/381; A61P 25/18

(57) ABSTRACT

Neuropsychiatric treatments, including methods, regimens, and interventions to reduce physical dependence to those neuropsychiatric treatments based on ulotaront administration.

### PRIORITY

The present application claims priority to provisional U.S. Application No. 63/379,364, filed Oct. 13, 2022, the contents of which are incorporated herein by reference in their entirety.

### FIELD

The present disclosure relates to pharmacological neuropsychiatric treatments, and to methods, regimens, and interventions to reduce physical dependence to those neuropsychiatric treatments based on ulotaront administration.

### BACKGROUND

Neuropsychiatric medications are a heterogeneous group of compounds with a high variability of receptor affinities. The clinical effects of the individual compounds are diverse, since receptor affinity is an important factor for the efficacy, but especially for the side effect profile. Correll (2010).

Physical dependence, which can develop from therapeutic use of neuropsychiatric medications, is a physical condition in which abrupt or gradual drug withdrawal causes adverse side effects. The appropriate discontinuation strategy is a complex issue and depends on the side effect profile of the drug, its pharmacodynamics and kinetics, and behavioral mechanisms, as well as comorbidities and vulnerabilities of the patient. Cerovecki et al. (2013).

Ulotaront (a/k/a SEP-363856, SEP-856) is an investigational medication under clinical development by Sunovion Pharmaceuticals Inc. (Marlborough, Massachusetts) for various neurological disorders. In animal testing, ulotaront has demonstrated a predominantly antipsychotic-like signature in addition to anxiolytic and antidepressant components. Dedic et al. (2019). A physical dependence study in rats is reported in US 2021/0315859 A1.

Abruptly stopping neuropsychiatric therapy comes with a high risk of withdrawal symptoms that include a variety of somatic, motor, and psychological symptoms. Chouinard et al. (2017). Depending on the medication's target receptor, both first and second generation antipsychotics can be associated with the following clinical features when the medication is withdrawn or reduced: cholinergic withdrawal symptoms such as agitation, insomnia, anxiety or depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion or disorientation, hypothermia, and sweating; dopaminergic withdrawal symptoms (nigrostriatal) selected from withdrawal dyskinesia, parkinsonism, neuroleptic malignant syndrome, and akathisia; serotonin withdrawal symptoms selected from flu-like symptoms, sweating or chills, dizziness, light-headedness or tachycardia, paresthesia, electric chock sensations, anxiety, agitation, low mood, insomnia, nightmares, nausea, vomiting, diarrhea, confusion, and decreased concentration; histaminergic withdrawal symptoms selected from irritability, insomnia, agitation, depressed affect, loss of appetite or nausea, tremulousness, incoordination, and lethargy or amnesia; dopaminergic withdrawal symptoms (mesolimbic or striatal) selected from auditory hallucinations, persecutory delusions, and other psychotic symptoms; and adrenergic withdrawal symptoms selected from headache, anxiety or agitation, hypertension, tachycardia, angina, palpitations, risk of myocardial infarction, pre-syncope, tremulousness, and sweating. Horowitz et al. (2021).

Patients who show tolerance to dose increases are more likely to have withdrawal symptoms. Chouinard et al. (2017). Research also shows that the longer the exposure to medication the higher the risk of developing withdrawal-associated psychosis on discontinuation. Tiihonen et al. (2018).

Relapse is another significant risk from neuropsychiatric medication discontinuation. One study reported that 48% of relapses occur in the first 12 months after discontinuation (40% in the first 6 months), with only 2% per year after this period. Viguera et al. (1997). The risk of relapse doubles after 1-2 years, triples after 2-5 years, and increases 7 times after 8 years of medication exposure. Horowitz et al. (2021) recently published a method for tapering neuropsychiatric medications to minimize the risk of relapse, done gradually over months, even years, incorporating hyperbolic dose reductions to provide a more even reduction of D2 blockade.

What is needed are neuropsychiatric regimens, and withdrawal strategies, that do not give rise to the problems historically associated with withdrawal from neuropsychiatric treatments.

### SUMMARY

It has unexpectedly been discovered that ulotaront has an excellent side effect profile when human subjects are withdrawn from the drug, and that subjects may be withdrawn from ulotaront therapy either abruptly or gradually without significant risk of many, if not all, clinically significant complications.

Therefore, in one embodiment, the disclosure provides a method of treating a neuropsychiatric disorder in a human subject in need thereof without significant physical dependency upon the cessation of treatment, comprising selecting and administering a therapeutically effective amount of ulotaront or a pharmaceutically acceptable salt thereof to the subject for an effective period until ceasing administration, whereupon significant physical dependency greater than placebo does not occur.

In another embodiment, the disclosure provides a method of treating a neuropsychiatric disorder in a human subject in need thereof, comprising: (a) administering to the subject a therapeutically effective amount of ulotaront or a pharmaceutically acceptable salt thereof for an effective period, and (b) ceasing ulotaront administration abruptly after the effective period.

In another embodiment, the disclosure provides a method of treating a neuropsychiatric disorder in a human subject in need thereof, comprising: (a) administering to the subject a therapeutically effective amount of ulotaront or a pharmaceutically acceptable salt thereof for an effective period, and (b) after the effective period, ceasing ulotaront administration and any pharmaceutical therapy for the neuropsychiatric disorder.

In another embodiment, the disclosure provides a method of treating a neuropsychiatric disorder in a human subject in need thereof, comprising: (a) administering to the subject a therapeutically effective amount of ulotaront or a pharmaceutically acceptable salt thereof for an effective period, and (b) ceasing ulotaront administration after the effective period; wherein the subject is at risk of, or the subject has previously experienced, or the method occurs without emergence of, one or more symptom(s) or syndrome(s) of neuropsychiatric medication withdrawal.

Additional advantages of the disclosure are set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

All published documents cited herein are hereby incorporated herein by reference in their entirety.

### Use of Terms

As used herein, the singular forms “a”, “an” and “the” are intended to include the plural forms as well, unless the context clearly indicates otherwise.

Unless otherwise specified, the word “includes” (or any variation thereon, e.g., “include”, “including”, etc.) is intended to be open-ended. For example, “A includes 1, 2 and 3” means that A includes, but is not limited to, 1, 2 and 3.

As used in this specification and in the claims which follow, the word “comprise” and variations of the word, such as “comprising” and “comprises,” means “including but not limited to,” and is not intended to exclude, for example, other additives, components, integers or steps.

When an element is described as comprising a plurality components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or particular numerical values are specified, it will be understood that a range can be defined by selectively combining any of the lower end variables, upper end variables, and particular numerical values that is mathematically possible. When ranges are stated as extending from one endpoint to another endpoint, it will be understood that the two endpoints are included in the range. However, it will also be understood that a from/to range also includes an embodiment in which the range is defined as between the two specified endpoints, and that the term “between” can be substituted for the “from/to” language to omit the endpoints from the range.

The present disclosure describes various embodiments. A person of ordinary skill in the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

When published test methodologies and diagnostic instruments are referred to herein, it will be understood that the test methodology or diagnostic instrument is performed based on the version in effect on Oct. 1, 2022, unless otherwise stated to the contrary herein. This is true even when the methodology or instrument is defined herein based on a publication reporting an earlier version.

Unless defined otherwise, all technical and scientific terms used herein have the same meaning as commonly understood by one of ordinary skill in the art to which this disclosure belongs.

### Definitions

“Adjunctive MDD therapy” or “AMDD therapy” refers to the addition of an agent to an antidepressant regimen in order to improve efficacy, because the subject has experienced an inadequate response to an antidepressant regimen that has been optimized in dose and duration.

As used herein, “administering” or “administration” of ulotaront, or a pharmaceutically acceptable salt thereof, encompasses the delivery to a subject of ulotaront, or a pharmaceutically acceptable salt thereof, or a prodrug or other pharmaceutically acceptable derivative thereof, using any suitable formulation or route of administration, e.g., as described herein.

An “AE” or “adverse event” is any untoward medical occurrence associated with the use of a drug in humans, whether or not considered drug related. Those untoward medical occurrences that occur after first administration of study drug are considered AEs. An AE can, therefore, be any unfavorable and unintended sign (including an abnormal laboratory finding), symptom, or disease occurring after the administration of a medicinal (investigational) product, whether or not considered related to the medicinal (investigational) product. AEs may include the onset of new illness and the exacerbation of pre-existing conditions. A “mild” AE, as used herein and throughout this document, refers to “ordinarily transient symptoms that do not influence performance of subject's daily activities. Other treatment is not ordinarily indicated.” A “moderate” AE, as used herein and throughout this document, refers to “marked symptoms sufficient to make the subject uncomfortable. Moderate influence on performance of subject's daily activities. Other treatment may be necessary.” A “severe” AE, as used herein and throughout this document, refers to “symptoms that cause considerable discomfort. Substantial influence on subject's daily activities. May be unable to continue the study, and other treatment may be necessary.”

As disclosed herein, the methods of the current disclosure can be undertaken without occurrence or emergence of one or more adverse events selected from symptom(s) or syndrome(s) of neuropsychiatric medication withdrawal, typically defined based on a Markush grouping of adverse events. By “one or more” is meant that the methods can be practiced without the occurrence or emergence of any of the recited adverse events, that the methods can be practiced without the occurrence of emergence of any particular one of the recited adverse events, or that the methods can be practiced without the occurrence or emergence of any combination of the recited adverse events. Consistent with Markush groupings practice, it will be understood that the Markush grouping can be limited to only one species within the grouping.

When a drug treatment is said to occur without inducing physical dependence, or cessation of drug treatment is said to occur without adverse events, or symptoms or syndromes of medication withdrawal, it will be understood to refer only to physical dependence, adverse events, or symptoms or syndromes of medication withdrawal which in the clinical judgment of a treating physician are significant and resulting from the drug treatment or cessation thereof. In one embodiment, “significant” refers to a moderate or severe adverse event.

The “AIMS” (Abnormal Involuntary Movement Scale) assessment consists of 10 items describing symptoms of dyskinesia. Guy 1976. Facial and oral movements (items 1 through 4), extremity movements (items 5 and 6), and trunk movements (item 7) are observed unobtrusively while the subject is at rest; the investigator also makes global judgments on the subject's dyskinesias (items 8 through 10). Each item is rated on a 5-point scale, with a score of zero representing absence of symptoms (for item 10, no awareness), and a score of 4 indicating a severe condition (for item 10, awareness, severe distress). In addition, the AIMS includes 2 yes/no questions that address the subject's dental status. The AIMS Movement Rating Score is defined as the sum of items 1 through 7 (i.e., items 1 through 4, facial and oral movements; items 5 and 6, extremity movements; and item 7, trunk movements).

An “antidepressant regimen” or “antidepressant therapy” refers to any pharmacological therapeutic regimen administered as therapy for the treatment of depression, and should be distinguished from augmentation, which is the addition of an agent—not thought to be an antidepressant itself—to an antidepressant regimen in order to improve efficacy. Examples of pharmacological agents for the treatment of depression according to this disclosure include, without limitation, SSRIs, SNRIs, bupropion and mirtazapine, and their pharmaceutically acceptable salts.

As used herein, an “at risk” individual is an individual who is at risk of developing a disorder to be treated or an adverse event to be prevented. This may be shown, for example, by one or more risk factors, which are measurable parameters that correlate with development of a disorder and are known in the art. When a method is said to treat a condition without causing or eliciting an adverse event, it will be understood that the methods can be performed in subjects at risk of the adverse event.

The “BARS” (Barnes Akathisia Rating Scale) is a global clinical assessment of akathisia. Barnes 1989. The BARS consists of 4 items related to akathisia: objective observation of akathisia by the investigator, subjective feelings of restlessness by the subject, subjective distress due to akathisia, and global clinical assessment of akathisia. The first 3 items are rated on a 4-point scale, with a score of zero representing absence of symptoms and a score of 3 representing a severe condition. The global clinical evaluation is made on a 6-point scale, with zero representing absence of symptoms and a score of 5 representing severe akathisia.

The “CGI-C” (Clinical Global Impression—Change) Scale measures efficacy of drug treatment by rating the subject's total change from the initiation of drug therapy whether or not it is due entirely to drug treatment. Response choices include: 1=very much improved, 2=much improved, 3=minimally improved, 4=no change, 5=minimally worse, 6=much worse, and 7=very much worse.

The “CGI-S” (Clinical Global Impression—Severity) Scale is a standardized, clinician-administered global rating scale that measures disease severity on a 7-point Likert scale. A higher score on the CGI-S represents a higher severity of disease. To perform this assessment, the rater or investigator answers the following question: “Considering your total clinical experience with this particular population, how mentally ill is the patient at this time?” Response choices include: 1=normal, not at all ill; 2=borderline mentally ill; 3=mildly ill; 4=moderately ill; 5=markedly ill; 6=severely ill; and 7=among the most extremely ill patients.

As used herein, a “clinically significant” or “clinically meaningful” improvement can mean an improvement which is both statistically significant, and meaningful from a patient's, clinician's, or caregiver's perspective, typically based on a static measure such as CGI-S or a retrospective evaluation of improvement such as the CGI-C, as described generally in various publications of the United States Food and Drug Administration, including FDA 2018, FDA 2019, and FDA 2020. When a treatment or benefit is described herein, it will be understood that the treatment or benefit preferably shows clinically significant efficacy in a population of patients to a degree of statistical significance.

The Columbia-Suicide Severity Rating Scale (C-SSRS) is a tool designed to systematically assess and track suicidal AEs (suicidal behavior and suicidal ideation) throughout the trial. The strength of this suicide classification system is in its ability to comprehensively identify suicidal events while limiting the over-identification of suicidal behavior. Nilsson et al. (2013). The scale takes approximately 5 minutes to administer.

As used herein, “delaying” development of a disorder means to defer, hinder, slow, stabilize, and/or postpone development of the disorder. Delay can be of varying lengths of time, depending on the history of the disease and/or the individual being treated.

“Depression” has the meaning normally ascribed to the term in the field of psychiatric medicine and can assume the definition set forth in DSM-5. When depression is referred to herein, it will be understood that Major Depressive Disorder (“MDD”) is a species of depression, and that the disclosure in all aspects is more specifically targeted at the treatment of MDD, particularly as AMDD.

“DSM-5” refers to the Diagnostic and Statistical Manual of Mental Disorders, Fifth Edition. Terms used herein can be defined by reference to DSM-5 when necessary to give life and meaning to the term. When a person is defined in this document based on DSM-5, it will be understood that the person need not have been diagnosed using the criteria in DSM-5, but that the person would meet the criteria specified in DSM-5 is so diagnosed.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of ulotaront include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions (X) are quite acceptable as synthetic intermediates. Thus, X may be pharmaceutically undesirable anions, such as iodide, oxalate, trifluoromethanesulfonate and the like, when such salts are chemical intermediates.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, which has been approved by or is otherwise acceptable to the United States Food and Drug Administration upon proper qualification for use in humans or domestic animals.

The Physician Withdrawal Checklist (PWC) is a 34-item physician-administered questionnaire that was developed for the evaluation of benzodiazepine withdrawal symptoms. The PWC 20 total score includes 20 items from the PWC that have been validated for internal consistency, test retest and inter-rater reliability, and factor structure. Rickels (2008).

As used herein, “prevention” or “preventing” refers to a regimen that protects against the onset of the disorder such that the clinical symptoms of the disorder do not develop. Accordingly, “prevention” relates to administration of a therapy to a subject before signs of the diseases are detectable in the subject (for example, administration of a therapy in the absence of a detectable symptom of the disorder). The subject may be an individual at risk of developing the disorder.

The “SAS” (Simpson Angus Scale) consists of a list of 10 symptoms of Parkinsonism (gait, arm dropping, shoulder shaking, elbow rigidity, wrist rigidity, head rotation, glabella tap, tremor, salivation, and akathisia). Simpson 1970. Each item is rated on a 5-point scale, with a score of zero representing absence of symptoms, and a score of 4 representing a severe condition. The SAS Total Score is the sum of the scores for all 10 items.

The term “selecting” refers to the act of choosing from a number or group by fitness or preference. In the context of the current disclosure, ulotaront is selected from a group of generally recognized neuropsychiatric medications, for the treatment of any of the neuropsychiatric conditions described herein, based on the lack of physical dependence induced by ulotaront, whether by short- or long-term treatment, or mild, moderate or intensive treatment.

As used herein, the term “significantly” refers to a level of statistical significance. The level of statistical significance can be p<0.1, p<0.05, p<0.01, p<0.005, or p<0.001. Unless otherwise specified, the level of statistical significance when the term “significant,” “significantly,” or other variations of the term are used is p<0.05. When a measurable result or effect is expressed or identified herein, it will be understood that the result or effect is preferably evaluated based upon its statistical significance relative to a baseline such as placebo. In like manner, when a treatment or benefit is described herein, it will be understood that the treatment or benefit preferably shows efficacy in a population of patients to a degree of statistical significance.

“SNRIs” (Serotonin-norepinephrine Reuptake Inhibitors) include, without limitation, desvenlafaxine (Pristiq®), duloxetine (Cymbalta®), levomilnacipran (Fetzima®), and venlafaxine (Effexor® XR), and their pharmaceutically acceptable salts.

“SSRIs” (Selective Serotonin Reuptake Inhibitors) include, without limitation, citalopram (Celexa®), escitalopram (Lexapro®), fluoxetine (Prozac®), paroxetine (Paxil®, Pexeva®), and sertraline (Zoloft®), and their pharmaceutically acceptable salts.

As used herein, “subject” or “patient,” which terms are used interchangeably, to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, the term “therapeutically effective amount” or “effective amount” refers to an amount that is effective to elicit the desired biological or medical response, including the amount of a compound that, when administered to a subject for treating a disorder, is sufficient to accomplish such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, the terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed, for example, acute exacerbation of symptoms. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

When treatment with two separate agents is described herein, it will be understood that the agents will be administered concomitantly, based on concurrently running dosing regimens, which may differ in dosing frequency or time of administration. The two agents do not necessarily have to be administered at the same time for the administration to be concomitant. For example, concomitant administration may include one agent that is administered three times per day and one agent that is administered once a day.

Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder.

In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

“Ulotaront,” as referred to herein for use in the methods of the present disclosure, has the chemical name (S)-(4,5-dihydro-7H-thieno[2,3-c]pyran-7-yl)-N-methylmethanamine (which may be abbreviated as “(S)-TPMA”). Ulotaront has the following structure:

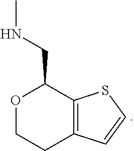

Unless stated otherwise, or unless context requires otherwise, for purposes of this disclosure, the term “ulotaront,” standing alone, includes the free form of ulotaront and also includes its pharmaceutically acceptable salts, hydrates, solvates, amorphous and crystalline forms. When the free form is intended, or any other form or salt is specifically intended, it will be stated as such expressly.

Ulotaront can be used in the methods described herein as the free base or in the form of a pharmaceutically acceptable salt. In preferred embodiments, a hydrochloric acid (HCl) salt of ulotaront is used in the methods described herein. Ulotaront, or a pharmaceutically acceptable salt thereof, including its HCl crystalline forms, can be obtained according to the production methods described in PCT Patent Publication No. WO2011/069063 (U.S. Pat. No. 8,710,245, issued Apr. 29, 2014) or PCT Patent Publication No. WO2019/161238, which are incorporated herein by reference in entirety and for all purposes, or a method analogous thereto.

Methods of using ulotaront for the treatment of the neuropsychiatric disorders described herein, particularly schizophrenia, depression, and anxiety, are described in PCT Patent Publication No. WO 2020/118032 and Dedic et al. (2019).

Also provided herein are pharmaceutical compositions and dosage forms, comprising ulotaront, or a pharmaceutically acceptable salt thereof, and one or more pharmaceutically acceptable excipients. Compositions and dosage forms provided herein may further comprise one or more additional active ingredients. Ulotaront, or a pharmaceutically acceptable salt thereof, may be administered as part of a pharmaceutical composition as described herein.

### DISCUSSION

Physical dependence, which can develop from therapeutic use of neuropsychiatric medications, produces a high risk of withdrawal symptoms that include a variety of somatic, motor, and psychological symptoms. The disclosure relates to the act of selecting ulotaront from among available neuropsychiatric medications, and a consequent reduction in physical dependence and withdrawal symptoms when therapy is ceased.

In one embodiment, the disclosure provides a method of treating a neuropsychiatric disorder in a human subject in need thereof without significant physical dependency upon the cessation of treatment, comprising selecting and administering a therapeutically effective amount of ulotaront or a pharmaceutically acceptable salt thereof to the subject for an effective period until ceasing administration, whereupon significant physical dependency greater than placebo does not occur. In one embodiment, the administration is daily.

In another embodiment, the disclosure provides a method of treating a neuropsychiatric disorder in a human subject in need thereof, comprising: (a) administering to the subject a therapeutically effective amount of ulotaront or a pharmaceutically acceptable salt thereof for an effective period, and (b) ceasing ulotaront administration abruptly after the effective period. In one embodiment, the method is undertaken without significant physical dependency upon cessation of treatment in a subject in need thereof whereupon significant physical dependency greater than placebo does not occur.

In another embodiment, the disclosure provides a method of treating a neuropsychiatric disorder in a human subject in need thereof, comprising: (a) administering to the subject a therapeutically effective amount of ulotaront or a pharmaceutically acceptable salt thereof for an effective period, and (b) after the effective period, ceasing ulotaront administration and any pharmaceutical therapy for the neuropsychiatric disorder. In one embodiment, the method is undertaken without significant physical dependency upon cessation of treatment in a subject in need thereof whereupon significant physical dependency greater than placebo does not occur.

In another embodiment, the disclosure provides a method of treating a neuropsychiatric disorder in a human subject in need thereof, comprising: (a) administering to the subject a therapeutically effective amount of ulotaront or a pharmaceutically acceptable salt thereof for an effective period, and (b) ceasing ulotaront administration after the effective period; wherein the subject is at risk of, or the subject has previously experienced, or the method occurs without emergence of, one or more symptom(s) or syndrome(s) of neuropsychiatric medication withdrawal.

In one embodiment, the method is undertaken without significant physical dependency upon cessation of treatment in a subject in need thereof whereupon significant physical dependency greater than placebo does not occur.

The method can be used to treat many neuropsychiatric disorders, symptoms of those disorders, or side effects associated with the conventional treatment of these disorders.

Representative disorders include schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, aggression, delirium, Parkinson's psychosis, excitative psychosis, Tourette's syndrome, organic or NOS psychosis, seizure, agitation, post-traumatic stress disorder, behavior disorder, neurodegenerative disease, Alzheimer's disease, Parkinson's disease, dyskinesias, Huntington's disease, dementia, mood disorder, anxiety, affective disorders (e.g., depression, e.g., major depressive disorder and dysthymia; bipolar disorder, e.g., bipolar depressive disorder; manic disorder; seasonal affective disorder; and attention deficit disorder (ADD) and attention deficit hyperactivity disorder (ADHD)), obsessive-compulsive disorder, vertigo, epilepsy, pain (e.g., neuropathic pain, sensitization accompanying neuropathic pain, and inflammatory pain), fibromyalgia, migraine, cognitive impairment, movement disorder, restless leg syndrome (RLS), multiple sclerosis, sleep disorder, sleep apnea, narcolepsy, excessive daytime sleepiness, jet lag, drowsy side effect of medications, insomnia, substance abuse or dependency (e.g., nicotine, cocaine), addiction, eating disorder, sexual dysfunction, hypertension, emesis, Lesche-Nyhane disease, Wilson's disease, autism, Huntington's chorea, and premenstrual dysphoria.

Given ulotaront's unique behavioral signatures (as reported by Dedic 2019), the human clinical results reported in PCT Patent Publication No. WO 2020/118032, and ulotaront's novel mechanism of action and receptor binding profile, ulotaront can be expected to treat neuropsychiatric disorders with a high degree of safety and efficacy. Thus, in one embodiment, ulotaront is used to improve symptoms in neuropsychiatric disorders selected from psychoses, depression, pain, cognition, mood disorders, and anxiety.

In another embodiment, the neuropsychiatric disorders include schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, excitative psychosis, organic or NOS psychosis, dyskinesias, mood disorder, anxiety, affective disorders (e.g., depression, e.g., major depressive disorder and dysthymia; bipolar disorder, e.g., bipolar depressive disorder; manic disorder; seasonal affective disorder), pain (e.g., neuropathic pain, sensitization accompanying neuropathic pain, and inflammatory pain), cognitive impairment, movement disorder.

In another embodiment, the treatment is undertaken without inducing a clinically significant occurrence of conditions traditionally associated with neuropsychiatric treatments, including the treatment of such conditions, such as hyperprolactinaemia, blood prolactin abnormal, blood prolactin increased, galactorrhoea, cogwheel rigidity, obesity, metabolic syndrome, dyslipidemia, akathisia, extrapyramidal disorder, restlessness, increased appetite, pancreatitis chronic, weight increased, and nuchal rigidity.

In one embodiment, the neuropsychiatric disorder is selected from schizophrenia, depression, and anxiety. In another embodiment the neuropsychiatric disorder is schizophrenia.

In another embodiment the neuropsychiatric disorder is bipolar depression (particularly MDD as defined by DSM-5 and more particularly AMDD). In still another embodiment the neuropsychiatric disorder is anxiety (for example, generalized anxiety disorder (GAD) as defined by DSM-5).

The “effective period” is a period of time adequate to judge the efficacy or ineffectiveness of the treatment. Thus, in various embodiments, the effective period is a term≥2 weeks, ≥4 weeks, ≥8 weeks, ≥12 weeks, ≥26 weeks, ≥one year, or ≥two years, or optionally less than 5 years, 4 years, or 3 years.

In other embodiments, the effective period is described functionally. Thus, in one embodiment, the effective period is sufficient for a clinically significant improvement from the neuropsychiatric disorder. In another embodiment, the effective period is sufficient for full recovery from the neuropsychiatric disorder. In other embodiments, the effective period is sufficient for full recovery from the neuropsychiatric disorder, as evidenced by a lack of diagnosis of the neuropsychiatric disorder under criteria set forth in DSM-5. In still other embodiments, the effective period is sufficient for full recovery from the neuropsychiatric disorder for a term≥3 months, ≥6 months, ≥1 year, ≥18 months, or ≥2 years.

The effective period can also be the period of time for judging ulotaront's ineffectiveness. Thus, in one embodiment the effective period is sufficient to judge the ineffectiveness of ulotaront against the subject's neuropsychiatric disorder. In other embodiments, the effective period is sufficient to judge the ineffectiveness of ulotaront against the subject's neuropsychiatric disorder, as evidenced by the failure of a clinically significant improvement from the neuropsychiatric disorder. In other embodiments, the effective period is sufficient to judge the ineffectiveness of ulotaront against the subject's neuropsychiatric disorder, as evidenced by a continued diagnosis of the neuropsychiatric disorder under criteria set forth in DSM-5. In still further embodiments, the effective period is sufficient to judge the ineffectiveness of ulotaront against the subject's neuropsychiatric disorder, as evidenced by one or more of: (a) inadequate clinical response for acute symptoms despite dose optimization and adequate duration of treatment trial; (b) poor control of chronic symptoms and persistence of functional disabilities during maintenance therapy; (c) relapse despite adequate prophylactic or maintenance treatment of the neuropsychiatric disorder; and (d) persistence of certain symptoms of the neuropsychiatric disorder despite adequate doses of ulotaront.

The methods can also be defined by the nature of cessation of ulotaront therapy. The cessation will either be abrupt (i.e., without any tapering) or with tapering. By “tapering” is meant any step-wise reduction in the dose, at any frequency over any period of time. In some embodiments, the dose reduction occurs in increments of 12.5 and/or 25 mg. In some embodiments, the dose reduction and eventual cessation occurs over a period of from one week to one year. Step-wise does not mean that the reductions must be equal in degree. Thus, for example, a stepwise reduction would include the hyperbolic dose reductions proposed by Horowitz (2021).

In any of the embodiments, the subject can be at risk of one or more symptom(s) or syndrome(s) of neuropsychiatric medication withdrawal. Alternatively, in any of the embodiments the subject can have previously experienced one or more symptom(s) or syndrome(s) of neuropsychiatric medication withdrawal. In still further alternatives, applicable to any of the embodiments, the method occurs without emergence of one or more symptom(s) or syndrome(s) of neuropsychiatric medication withdrawal. In still further alternatives, significant physical dependency is defined as the occurrence or emergence of one or more symptom(s) or syndrome(s) of neuropsychiatric medication withdrawal.

In some embodiments, the subject can be at risk of, or the subject can have previously experienced, or the method can occur without emergence of, a withdrawal-related adverse event comprising a clinically significant increase in the total score of the 20 item Physician Withdrawal Checklist (PWC-20). In other embodiments, significant physical dependency is defined as the occurrence or emergence of a withdrawal-related adverse event comprising a clinically significant increase in the total score of the 20 item Physician Withdrawal Checklist (PWC-20).

In some embodiments, the subject can be at risk of, or the subject can have previously experienced, or the method can occur without emergence of, a withdrawal-related adverse event comprising an increase in the total score of the 20 item Physician Withdrawal Checklist (PWC-20) of more than 4.0, 4.25. 4.5, 4.71, 4.75, 5.0, or 5.25. In other embodiments, significant physical dependency is defined as the occurrence or emergence of a withdrawal-related adverse event comprising an increase in the total score of the 20 item Physician Withdrawal Checklist (PWC-20) of more than 4.0, 4.25. 4.5, 4.71, 4.75, 5.0, or 5.25.

In some embodiments, the subject can be at risk of, or the subject can have previously experienced, or the method can occur without emergence of, a withdrawal-related adverse event defined by Standardized Medical Dictionary for Regulatory Activities (MedDRA) Query: Drug withdrawal. In other embodiments, significant physical dependency is defined as the occurrence or emergence of a withdrawal-related adverse event defined by Standardized Medical Dictionary for Regulatory Activities (MedDRA) Query: Drug withdrawal.

In some embodiments, the subject can be at risk of, or the subject can have previously experienced, or the method can occur without emergence of, a withdrawal-related adverse event comprising one or more symptom(s) of neuropsychiatric medication withdrawal selected from somatic symptoms, mood symptoms, cognitive symptoms, fatigue symptoms, and gastrointestinal symptoms. In other embodiments, significant physical dependency is defined as the occurrence or emergence of a withdrawal-related adverse event comprising one or more symptom(s) of neuropsychiatric medication withdrawal selected from somatic symptoms, mood symptoms, cognitive symptoms, fatigue symptoms, and gastrointestinal symptoms.

In some embodiments, the subject can be at risk of, or the subject can have previously experienced, or the method can occur without emergence of, a withdrawal-related adverse event comprising one or more somatic symptom(s) of neuropsychiatric medication withdrawal selected from (i) insomnia, (ii) diaphoresis, (iii) tremor-tremulousness, (iv) headaches, and (v) muscle aches or stiffness. In other embodiments, significant physical dependency is defined as the occurrence or emergence of a withdrawal-related adverse event comprising one or more somatic symptom(s) of neuropsychiatric medication withdrawal selected from (i) insomnia, (ii) diaphoresis, (iii) tremor-tremulousness, (iv) headaches, and (v) muscle aches or stiffness.

In some embodiments, the subject can be at risk of, or the subject can have previously experienced, or the method can occur without emergence of, a withdrawal-related adverse event comprising one or more mood symptom(s) of neuropsychiatric medication withdrawal selected from (i) anxiety-nervousness, (ii) irritability, (iii) dysphoric mood-depression, (iv) restlessness-agitation, and (v) difficulty concentrating, remembering. In other embodiments, significant physical dependency is defined as the occurrence or emergence of a withdrawal-related adverse event comprising one or more mood symptom(s) of neuropsychiatric medication withdrawal selected from (i) anxiety-nervousness, (ii) irritability, (iii) dysphoric mood-depression, (iv) restlessness-agitation, and (v) difficulty concentrating, remembering.

In some embodiments, the subject can be at risk of, or the subject can have previously experienced, or the method can occur without emergence of, a withdrawal-related adverse event comprising one or more cognitive symptom(s) of neuropsychiatric medication withdrawal selected from (i) poor coordination, (ii) dizziness-lightheadedness, (iii) increased acuity for sound, smell, touch, and (iv) depersonalization-derealization. In other embodiments, significant physical dependency is defined as the occurrence or emergence of a withdrawal-related adverse event comprising one or more cognitive symptom(s) of neuropsychiatric medication withdrawal selected from (i) poor coordination, (ii) dizziness-lightheadedness, (iii) increased acuity for sound, smell, touch, and (iv) depersonalization-derealization.

In some embodiments, the subject can be at risk of, or the subject can have previously experienced, or the method can occur without emergence of, a withdrawal-related adverse event comprising one or more fatigue symptom(s) of neuropsychiatric medication withdrawal selected from (i) fatigue-lethargy-lack of energy, (ii) weakness, and (iii) paresthesias. In other embodiments, significant physical dependency is defined as the occurrence or emergence of a withdrawal-related adverse event comprising one or more fatigue symptom(s) of neuropsychiatric medication withdrawal selected from (i) fatigue-lethargy-lack of energy, (ii) weakness, and (iii) paresthesias.

In some embodiments, the subject can be at risk of, or the subject can have previously experienced, or the method can occur without emergence of, a withdrawal-related adverse event comprising one or more gastrointestinal symptom(s) of neuropsychiatric medication withdrawal selected from (i) loss of appetite, (ii) nausea-vomiting, and (iii) diarrhea. In other embodiments, significant physical dependency is defined as the occurrence or emergence of a withdrawal-related adverse event comprising one or more gastrointestinal symptom(s) of neuropsychiatric medication withdrawal selected from (i) loss of appetite, (ii) nausea-vomiting, and (iii) diarrhea.

In any of the embodiments of the disclosure, the subject can be at risk of, or the subject can have previously experienced, or the method can occur without emergence of, a withdrawal-related adverse event comprising one or more withdrawal symptom(s) of mild, moderate, or severe magnitude. In other embodiments, significant physical dependency is defined as the occurrence or emergence of a withdrawal-related adverse event comprising one or more withdrawal symptom(s) of mild, moderate, or severe magnitude.

In like manner the subject can be at risk of, or the subject can have previously experienced, or the method can occur without emergence of, a withdrawal-related adverse event comprising one or more syndrome(s) of neuropsychiatric medication withdrawal selected from: (a) cholinergic syndrome evidenced by one or more of nausea, vomiting, headache, restlessness, anxiety, insomnia, fatigue, malaise, myalgia, diaphoresis, rhinitis, paresthesia, and loose bowels; (b) dopaminergic syndrome evidenced by one or more of withdrawal dyskinesia, akathisia, dystonia, tardive dyskinesia; and (c) rebound psychosis evidenced by one or more of psychosis above pre-treatment levels, illusions, hallucinations, and catatonia. In other embodiments, significant physical dependency is defined as the occurrence or emergence of a withdrawal-related adverse event comprising one or more syndrome(s) of neuropsychiatric medication withdrawal selected from: (a) cholinergic syndrome evidenced by one or more of nausea, vomiting, headache, restlessness, anxiety, insomnia, fatigue, malaise, myalgia, diaphoresis, rhinitis, paresthesia, and loose bowels; (b) dopaminergic syndrome evidenced by one or more of withdrawal dyskinesia, akathisia, dystonia, tardive dyskinesia; and (c) rebound psychosis evidenced by one or more of psychosis above pre-treatment levels, illusions, hallucinations, and catatonia.

In some embodiments, the subject can be at risk of, or the subject can have previously experienced, or the method can occur without emergence of, a withdrawal-related adverse event selected from cholinergic withdrawal symptoms, dopaminergic withdrawal symptoms (nigrostriatal), serotonin withdrawal symptoms, histaminergic withdrawal symptoms, dopaminergic withdrawal symptoms (mesolimbic or striatal), and adrenergic withdrawal symptoms. In other embodiments, significant physical dependency is defined as the occurrence or emergence of a withdrawal-related adverse event selected from cholinergic withdrawal symptoms, dopaminergic withdrawal symptoms (nigrostriatal), serotonin withdrawal symptoms, histaminergic withdrawal symptoms, dopaminergic withdrawal symptoms (mesolimbic or striatal), and adrenergic withdrawal symptoms.

In some embodiments, the subject can be at risk of, or the subject can have previously experienced, or the method can occur without emergence of, a withdrawal-related adverse event comprising cholinergic withdrawal symptoms selected from agitation, insomnia, anxiety or depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion or disorientation, hypothermia, and sweating. In other embodiments, significant physical dependency is defined as the occurrence or emergence of a withdrawal-related adverse event comprising cholinergic withdrawal symptoms selected from agitation, insomnia, anxiety or depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion or disorientation, hypothermia, and sweating.

In further embodiments, the subject can be at risk of, or the subject can have previously experienced, or the method can occur without emergence of, a withdrawal-related adverse event comprising dopaminergic withdrawal symptoms (nigrostriatal) selected from withdrawal dyskinesia, parkinsonism, neuroleptic malignant syndrome, and akathisia. In other embodiments, significant physical dependency is defined as the occurrence or emergence of a withdrawal-related adverse event comprising dopaminergic withdrawal symptoms (nigrostriatal) selected from withdrawal dyskinesia, parkinsonism, neuroleptic malignant syndrome, and akathisia.

In some embodiments, the subject can be at risk of, or the subject can have previously experienced, or the method can occur without emergence of, a withdrawal-related adverse event comprising serotonin withdrawal symptoms selected from flu-like symptoms, sweating or chills, dizziness, light-headedness or tachycardia, paresthesia, electric chock sensations, anxiety, agitation, low mood, insomnia, nightmares, nausea, vomiting, diarrhea, confusion, and decreased concentration. In other embodiments, significant physical dependency is defined as the occurrence or emergence of a withdrawal-related adverse event comprising serotonin withdrawal symptoms selected from flu-like symptoms, sweating or chills, dizziness, light-headedness or tachycardia, paresthesia, electric chock sensations, anxiety, agitation, low mood, insomnia, nightmares, nausea, vomiting, diarrhea, confusion, and decreased concentration.

Still further, the subject can be at risk of, or the subject can have previously experienced, or the method can occur without emergence of, a withdrawal-related adverse event comprising histaminergic withdrawal symptoms selected from irritability, insomnia, agitation, depressed affect, loss of appetite or nausea, tremulousness, incoordination, and lethargy or amnesia. In other embodiments, significant physical dependency is defined as the occurrence or emergence of a withdrawal-related adverse event comprising histaminergic withdrawal symptoms selected from irritability, insomnia, agitation, depressed affect, loss of appetite or nausea, tremulousness, incoordination, and lethargy or amnesia.

In addition, the subject can be at risk of, or the subject can have previously experienced, or the method can occur without emergence of, a withdrawal-related adverse event comprising dopaminergic withdrawal symptoms (mesolimbic or striatal) selected from auditory hallucinations, persecutory delusions, and other psychotic symptoms. In other embodiments, significant physical dependency is defined as the occurrence or emergence of a withdrawal-related adverse event comprising dopaminergic withdrawal symptoms (mesolimbic or striatal) selected from auditory hallucinations, persecutory delusions, and other psychotic symptoms.

In other embodiment, the subject can be at risk of, or the subject can have previously experienced, or the method can occur without emergence of, a withdrawal-related adverse event comprising adrenergic withdrawal symptoms selected from headache, anxiety or agitation, hypertension, tachycardia, angina, palpitations, risk of myocardial infarction, pre-syncope, tremulousness, and sweating. In other embodiments, significant physical dependency is defined as the occurrence or emergence of a withdrawal-related adverse event comprising adrenergic withdrawal symptoms selected from headache, anxiety or agitation, hypertension, tachycardia, angina, palpitations, risk of myocardial infarction, pre-syncope, tremulousness, and sweating.

It will be understood that any of the foregoing embodiments can be limited to subjects at risk of one or more withdrawal-related adverse events. In like manner, it will be understood that any of the foregoing embodiments can be limited to subjects who have previously experienced one or more withdrawal-related adverse events. Still further, it will be understood that the method can occur without emergence of one or more withdrawal-related adverse events. Still further, the method can be undertaken without significant physical dependency.

The withdrawal-related adverse event can be characterized as mild, moderate, or severe magnitude. Thus, in any of the foregoing embodiments, the subject can be at risk of, or the subject can have previously experienced, or the method can occur without emergence of, a withdrawal-related adverse event of mild, moderate, or severe magnitude.

In one embodiment, the subject ceases all antipsychotic therapy for the neuropsychiatric disorder when ceasing ulotaront therapy. Thus, any of the foregoing embodiments can further comprise, after the effective period, ceasing ulotaront administration and any pharmaceutical therapy for the neuropsychiatric disorder, for a period≥3 months, ≥6 months, ≥1 year, ≥18 months, or ≥2 years.

The therapeutically effective amount of ulotaront can be described variously as 25-150 mg/day or 25-100 mg/day or 50-125 mg/day or 50-100 mg/day, administered orally. Alternatively, the therapeutically effective amount can be described as 25 mg/day, 50 mg/day, 75 mg/day, 100 mg/day, 125 mg/day, or 150 mg/day, orally administered. In any of the embodiments of the disclosure, the therapeutically effective amount can be administered once daily in the fed or fasted state. The ulotaront can also be administered as the hydrochloride salt.

As shown in Example 1, the incidence of post-dose AEs is compared between placebo and SEP-363856 treatment groups in subjects with schizophrenia. The Example 2 withdrawal study compares abrupt discontinuation of SEP-363856 (SEP-363856 switched to placebo) to continuous SEP-363856 treatment in subjects with schizophrenia.

Preferred aspects of the disclosure can be defined based on Embodiments AA through CD below:
[Embodiment AA]A method of treating a neuropsychiatric disorder in a human subject in need thereof without significant physical dependency upon the cessation of treatment comprising selecting and administering a therapeutically effective amount of ulotaront or a pharmaceutically acceptable salt thereof to the subject for an effective period until ceasing administration whereupon significant physical dependency greater than placebo does not occur.
[Embodiment AB]A method of treating a neuropsychiatric disorder in a human subject in need thereof, comprising:
a) administering to the subject a therapeutically effective amount of ulotaront or a pharmaceutically acceptable salt thereof for an effective period, and
b) ceasing ulotaront administration abruptly after the effective period.
[Embodiment AC]A method of treating a neuropsychiatric disorder in a human subject in need thereof, comprising:
a) administering to the subject a therapeutically effective amount of ulotaront or a pharmaceutically acceptable salt thereof for an effective period, and
b) after the effective period, ceasing ulotaront administration and any pharmaceutical therapy for the neuropsychiatric disorder.
[Embodiment AD]A method of treating a neuropsychiatric disorder in a human subject in need thereof, comprising:
a) administering to the subject a therapeutically effective amount of ulotaront or a pharmaceutically acceptable salt thereof for an effective period, and
b) ceasing ulotaront administration after the effective period;
   wherein the subject is at risk of, or the subject has previously experienced, or the method occurs without emergence of, one or more symptom(s) or syndrome(s) of neuropsychiatric medication withdrawal.
[Embodiment AE] The method of any one of embodiments AB-AD, undertaken without significant physical dependency upon the cessation of treatment in a subject in need thereof whereupon significant physical dependency greater than placebo does not occur.
[Embodiment AF] The method of any one of embodiments AA or AE, wherein significant physical dependency is defined as the occurrence of a withdrawal-related adverse event comprising a clinically significant increase in the total score of the 20 item Physician Withdrawal Checklist (PWC-20).
[Embodiment AG] The method of any one of embodiments AA or AE, wherein significant physical dependency is defined as the occurrence of a withdrawal-related adverse event comprising an increase in the total score of the 20 item Physician Withdrawal Checklist (PWC-20) of more than 4.71.
[Embodiment AH] The method of any one of embodiments AA or AE, wherein significant physical dependency is defined as the occurrence of a withdrawal-related adverse event defined by Standardized Medical Dictionary for Regulatory Activities (MedDRA) Query: Drug withdrawal.
[Embodiment A1] The method of any one of embodiments AA or AE, wherein significant physical dependency is defined as the occurrence of a withdrawal-related adverse event comprising one or more symptom(s) of neuropsychiatric medication withdrawal selected from somatic symptoms, mood symptoms, cognitive symptoms, fatigue symptoms, and gastrointestinal symptoms.
[Embodiment AJ] The method of any one of embodiments AA or AE, wherein significant physical dependency is defined as the occurrence of a withdrawal-related adverse event selected from cholinergic withdrawal symptoms, dopaminergic withdrawal symptoms (nigrostriatal), serotonin withdrawal symptoms, histaminergic withdrawal symptoms, dopaminergic withdrawal symptoms (mesolimbic or striatal), and adrenergic withdrawal symptoms.
[Embodiment AK] The method of any one of embodiments AA-AJ, wherein the effective period is a term≥2 weeks, ≥4 weeks, ≥8 weeks, ≥12 weeks, ≥26 weeks, ≥one year, or ≥two years.
[Embodiment AL] The method of any one of embodiments AA-AK, wherein the effective period is sufficient for a clinically significant improvement from the neuropsychiatric disorder.
[Embodiment AM] The method of any one of embodiments AA-AK, wherein the effective period is sufficient for full recovery from the neuropsychiatric disorder.
[Embodiment AN] The method of any one of embodiments AA-AK, wherein the effective period is sufficient for full recovery from the neuropsychiatric disorder, as evidenced by a lack of diagnosis of the neuropsychiatric disorder under criteria set forth in DSM-5.
[Embodiment AO] The method of any one of embodiments AA-AN, wherein the effective period is sufficient for full recovery from the neuropsychiatric disorder for a term≥3 months, ≥6 months, ≥1 year, ≥18 months, or ≥2 years.
[Embodiment AP] The method of any one of embodiments AA-AK, wherein the effective period is sufficient to judge the ineffectiveness of ulotaront against the subject's neuropsychiatric disorder.
[Embodiment AQ] The method of any one of embodiments AA-AK, wherein the effective period is sufficient to judge the ineffectiveness of ulotaront against the subject's neuropsychiatric disorder, as evidenced by the failure of a clinically significant improvement from the neuropsychiatric disorder.
[Embodiment AR] The method of any one of embodiments AA-AK, wherein the effective period is sufficient to judge the ineffectiveness of ulotaront against the subject's neuropsychiatric disorder, as evidenced by a continued diagnosis of the neuropsychiatric disorder under criteria set forth in DSM-5.
[Embodiment AS] The method of any one of embodiments AA-AK, AP, AQ, or AR, wherein the effective period is sufficient to judge the ineffectiveness of ulotaront against the subject's neuropsychiatric disorder, as evidenced by one or more of:
a) inadequate clinical response for acute symptoms despite dose optimization and adequate duration of treatment trial;
b) poor control of chronic symptoms and persistence of functional disabilities during maintenance therapy;
c) relapse despite adequate prophylactic or maintenance treatment of the neuropsychiatric disorder; and
d) persistence of certain symptoms of the neuropsychiatric despite adequate doses of ulotaront.
[Embodiment AT] The method of any one of embodiments AA-AS, wherein the ceasing is abrupt.
[Embodiment AU] The method of any one of embodiments AA or AC-AS, wherein the ceasing is tapered.
[Embodiment AV] The method of any one of embodiments AA-AU, wherein the subject is at risk of one or more symptom(s) or syndrome(s) of neuropsychiatric medication withdrawal.
[Embodiment AW] The method of any one of embodiments AA-AU, wherein the subject has previously experienced one or more symptom(s) or syndrome(s) of neuropsychiatric medication withdrawal.
[Embodiment AX] The method of any one of embodiments AA-AU, wherein the method occurs without emergence of one or more symptom(s) or syndrome(s) of neuropsychiatric medication withdrawal.
[Embodiment AY] The method of any one of embodiments AA-AU, wherein the subject is at risk of, or the subject has previously experienced, or the method occurs without emergence of, a withdrawal-related adverse event comprising a clinically significant increase in the total score of the 20 item Physician Withdrawal Checklist (PWC-20).
[Embodiment AZ] The method of any one of embodiments AA-AU, wherein the subject is at risk of, or the subject has previously experienced, or the method occurs without emergence of, a withdrawal-related adverse event comprising an increase in the total score of the 20 item Physician Withdrawal Checklist (PWC-20) of more than 4.71.
[Embodiment BA] The method of any one of embodiments AA-AU, wherein the subject is at risk of, or the subject has previously experienced, or the method occurs without emergence of, a withdrawal-related adverse event defined by Standardized Medical Dictionary for Regulatory Activities (MedDRA) Query: Drug withdrawal.
[Embodiment BB] The method of any one of embodiments AA-AU, wherein the subject is at risk of, or the subject has previously experienced, or the method occurs without emergence of, a withdrawal-related adverse event comprising one or more symptom(s) of neuropsychiatric medication withdrawal selected from somatic symptoms, mood symptoms, cognitive symptoms, fatigue symptoms, and gastrointestinal symptoms.
[Embodiment BC] The method of any one of embodiments AA-AU, wherein the subject is at risk of, or the subject has previously experienced, or the method occurs without emergence of, a withdrawal-related adverse event comprising one or more somatic symptom(s) of neuropsychiatric medication withdrawal selected from (i) insomnia, (ii) diaphoresis, (iii) tremor-tremulousness, (iv) headaches, and (v) muscle aches or stiffness.
[Embodiment BD] The method of any one of embodiments AA-AU, wherein the subject is at risk of, or the subject has previously experienced, or the method occurs without emergence of, a withdrawal-related adverse event comprising one or more mood symptom(s) of neuropsychiatric medication withdrawal selected from (i) anxiety-nervousness, (ii) irritability, (iii) dysphoric mood-depression, (iv) restlessness-agitation, and (v) difficulty concentrating, remembering.
[Embodiment BE] The method of any one of embodiments AA-AU, wherein the subject is at risk of, or the subject has previously experienced, or the method occurs without emergence of, a withdrawal-related adverse event comprising one or more cognitive symptom(s) of neuropsychiatric medication withdrawal selected from (i) poor coordination, (ii) dizziness-lightheadedness, (iii) increased acuity for sound, smell, touch, and (iv) depersonalization-derealization.
[Embodiment BF] The method of any one of embodiments AA-AU, wherein the subject is at risk of, or the subject has previously experienced, or the method occurs without emergence of, a withdrawal-related adverse event comprising one or more fatigue symptom(s) of neuropsychiatric medication withdrawal selected from (i) fatigue-lethargy-lack of energy, (ii) weakness, and (iii) paresthesias.
[Embodiment BG] The method of any one of embodiments AA-AU, wherein the subject is at risk of, or the subject has previously experienced, or the method occurs without emergence of, a withdrawal-related adverse event comprising one or more gastrointestinal symptom(s) of neuropsychiatric medication withdrawal selected from (i) loss of appetite, (ii) nausea-vomiting, and (iii) diarrhea.
[Embodiment BH] The method of any one of embodiments AA-AU, wherein the subject is at risk of, or the subject has previously experienced, or the method occurs without emergence of, a withdrawal-related adverse event comprising one or more withdrawal symptom(s) of mild, moderate, or severe magnitude.
[Embodiment BI] The method of any one of embodiments AA-AU, wherein the subject is at risk of, or the subject has previously experienced, or the method occurs without emergence of, a withdrawal-related adverse event comprising one or more syndrome(s) of neuropsychiatric medication withdrawal selected from:
a) cholinergic syndrome evidenced by one or more of nausea, vomiting, headache, restlessness, anxiety, insomnia, fatigue, malaise, myalgia, diaphoresis, rhinitis, paresthesia, and loose bowels;
b) dopaminergic syndrome evidenced by one or more of withdrawal dyskinesia, akathisia, dystonia, tardive dyskinesia; and
c) rebound psychosis evidenced by one or more of psychosis above pre-treatment levels, illusions, hallucinations, and catatonia.
[Embodiment BJ] The method of any one of embodiments AA-AU, wherein the subject is at risk of, or the subject has previously experienced, or the method occurs without emergence of, a withdrawal-related adverse event selected from cholinergic withdrawal symptoms, dopaminergic withdrawal symptoms (nigrostriatal), serotonin withdrawal symptoms, histaminergic withdrawal symptoms, dopaminergic withdrawal symptoms (mesolimbic or striatal), and adrenergic withdrawal symptoms.
[Embodiment BK] The method of any one of embodiments AA-AU, wherein the subject is at risk of, or the subject has previously experienced, or the method occurs without emergence of, a withdrawal-related adverse event comprising cholinergic withdrawal symptoms selected from agitation, insomnia, anxiety or depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion or disorientation, hypothermia, and sweating.
[Embodiment BL] The method of any one of embodiments AA-AU, wherein the subject is at risk of, or the subject has previously experienced, or the method occurs without emergence of, a withdrawal-related adverse event comprising dopaminergic withdrawal symptoms (nigrostriatal) selected from withdrawal dyskinesia, parkinsonism, neuroleptic malignant syndrome, and akathisia.
[Embodiment BM] The method of any one of embodiments AA-AU, wherein the subject is at risk of, or the subject has previously experienced, or the method occurs without emergence of, a withdrawal-related adverse event comprising serotonin withdrawal symptoms selected from flu-like symptoms, sweating or chills, dizziness, light-headedness or tachycardia, paresthesia, electric chock sensations, anxiety, agitation, low mood, insomnia, nightmares, nausea, vomiting, diarrhea, confusion, and decreased concentration.
[Embodiment BN] The method of any one of embodiments AA-AU, wherein the subject is at risk of, or the subject has previously experienced, or the method occurs without emergence of, a withdrawal-related adverse event comprising histaminergic withdrawal symptoms selected from irritability, insomnia, agitation, depressed affect, loss of appetite or nausea, tremulousness, incoordination, and lethargy or amnesia.
[Embodiment BO] The method of any one of embodiments AA-AU, wherein the subject is at risk of, or the subject has previously experienced, or the method occurs without emergence of, a withdrawal-related adverse event comprising dopaminergic withdrawal symptoms (mesolimbic or striatal) selected from auditory hallucinations, persecutory delusions, and other psychotic symptoms.
[Embodiment BP] The method of any one of embodiments AA-AU, wherein the subject is at risk of, or the subject has previously experienced, or the method occurs without emergence of, a withdrawal-related adverse event comprising adrenergic withdrawal symptoms selected from headache, anxiety or agitation, hypertension, tachycardia, angina, palpitations, risk of myocardial infarction, pre-syncope, tremulousness, and sweating.
[Embodiment BQ] The method of any one of embodiments AY-BP, wherein the subject is at risk of one or more withdrawal related adverse events.
[Embodiment BR] The method of any one of embodiments AY-BP, wherein the subject has previously experienced one or more withdrawal related adverse events.
[Embodiment BS] The method of any one of embodiments AY-BP, wherein the method occurs without emergence of one or more withdrawal related adverse events.
[Embodiment BT] The method of any one of embodiments AY-BP, wherein the subject is at risk of, or the subject has previously experienced, or the method occurs without emergence of, a withdrawal-related adverse event comprising one or more withdrawal symptom(s) of mild, moderate, or severe magnitude.
[Embodiment BU] The method of any one of embodiments AA-BT comprising, after the effective period, ceasing ulotaront administration and any pharmaceutical therapy for the neuropsychiatric disorder, for a period≥3 months, ≥6 months, ≥1 year, ≥18 months, or ≥2 years.
[Embodiment BV] The method of any one of embodiments AA-BU, wherein the therapeutically effective amount is 25-150 mg/day or 25-100 mg/day or 50-125 mg/day or 50-100 mg/day administered orally.
[Embodiment BW] The method of any one of embodiments AA-BU, wherein the therapeutically effective amount is 25 mg/day, 50 mg/day, 75 mg/day, 100 mg/day, 125 mg/day, or 150 mg/day, orally administered.
[Embodiment BX] The method of any one of embodiments AA-BW, wherein the therapeutically effective amount is administered once daily in the fed or fasted state.
[Embodiment BY] The method of any one of embodiments AA-BX, wherein the ulotaront is administered as the hydrochloride salt.
[Embodiment BZ] The method of any one of embodiments AA-BY, wherein the neuropsychiatric disorder is selected from schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, aggression, delirium, Parkinson's psychosis, excitative psychosis, Tourette's syndrome, organic or NOS psychosis, seizure, agitation, post-traumatic stress disorder, behavior disorder, neurodegenerative disease, Alzheimer's disease, Parkinson's disease, dyskinesias, Huntington's disease, dementia, mood disorder, anxiety, affective disorders (e.g., depression, e.g., major depressive disorder and dysthymia; bipolar disorder, e.g., bipolar depressive disorder; manic disorder; seasonal affective disorder; and attention deficit disorder (ADD) and attention deficit hyperactivity disorder (ADHD)), obsessive-compulsive disorder, vertigo, epilepsy, pain (e.g., neuropathic pain, sensitization accompanying neuropathic pain, and inflammatory pain), fibromyalgia, migraine, cognitive impairment, movement disorder, restless leg syndrome (RLS), multiple sclerosis, sleep disorder, sleep apnea, narcolepsy, excessive daytime sleepiness, jet lag, drowsy side effect of medications, insomnia, substance abuse or dependency (e.g., nicotine, cocaine), addiction, eating disorder, sexual dysfunction, hypertension, emesis, Lesche-Nyhane disease, Wilson's disease, autism, Huntington's chorea, and premenstrual dysphoria.
[Embodiment CA] The method of any one of embodiments AA-BY, wherein the neuropsychiatric disorder is selected from schizophrenia, depression, and anxiety.
[Embodiment CB] The method of any one of embodiments AA-BY, wherein the neuropsychiatric disorder is schizophrenia.
[Embodiment CC] The method of any one of embodiments AA-BY, wherein the neuropsychiatric disorder is selected from bipolar depression and major depression.
[Embodiment CD] The method of any one of embodiments AA-BY, wherein the neuropsychiatric disorder is anxiety.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the disclosure and are not intended to limit the scope of what the inventors regard as their disclosure.

### Example 1. Examination of Post-Dose Adverse Events (AEs) in Completed and Ongoing Studies of SEP-363856 in Schizophrenia Patients

An analysis was performed to examine post-dose adverse events (AEs) with a start date after the date of last treatment exposure in one completed double-blind, placebo-controlled study, one completed open-label study, and one ongoing (as of the date of this evaluation) open-label study of SEP-363856.

Post-dose AEs, defined as AEs with a start date after the date of last treatment exposure, were examined for the Withdrawal Population. However, as study drug was dosed at bedtime in the studies, AEs with a start date≥1 day after the last dose for patients in the Withdrawal Population who had ≥2 follow-up days were also examined. Results of these analyses are presented in Tables 1 and 2.

In the completed double-blind, placebo-controlled study, the incidence of post-dose AEs was similar between placebo and SEP-363856 treatment groups (9[23.7%] placebo; 8[23.5%] SEP-363856; Table 1). The only post-dose AE occurring in 82 patients in the SEP-363856 group was schizophrenia; incidence of this AE was similar between the treatment groups (4[0.5%] placebo; 4[11.8%] SEP-363856).

TABLE 1
Post-dose Adverse Events in the Completed, Double-Blind,
Placebo-Controlled Study: Withdrawal Population

| | | | SEP-363856 | |
|---|---|---|---|---|
| System Organ Class/ | Placebo | | 50 to 75 mg/d | |
| Preferred Term | (N = 38) | | (N = 34) | |
| Any Post-dose AE | 9 | (23.7%) | 8 | (23.5%) |
| Psychiatric disorders | 6 | (15.8%) | 6 | (17.6%) |
| Schizophrenia | 4 | (10.5%) | 4 | (11.8%) |
| Insomnia | 1 | (2.6%) | 1 | (2.9%) |

| | | | |
|---|---|---|---|
| Agitation | 1 | (2.6%) | 0 |

| Psychotic disorder | 0 | 1 | (2.9%) |
|---|---|---|---|
| Cardiac disorders | 0 | 1 | (2.9%) |
| Cardiovascular insufficiency | 0 | 1 | (2.9%) |
| General disorders and administration site | 0 | 1 | (2.9%) |
| conditions | | | |
| Pyrexia | 0 | 1 | (2.9%) |

| | | | |
|---|---|---|---|
| Investigations | 3 | (7.9%) | 0 |
| Weight increased* | 2 | (5.3%) | 0 |
| Alanine aminotransferase increased | 1 | (2.6%) | 0 |

Abbreviations: d = day, mg = milligrams
Note:
Withdrawal Population includes patients who took at least one dose of study medication and had at least one day of post-dose follow-up time. Rollover patients in double-blind studies, and ongoing subjects are excluded from the withdrawal population.
Note:
n is the number of patients with events. Percentages are based on the total number of patients in the treatment group in the Withdrawal Population. Subjects are counted once per preferred term and system organ class.
Note:
Post-dose Adverse Events (AEs) are AEs with a start date after the date of last treatment exposure.

In completed and ongoing open-label studies, as of the date of this evaluation, a total of 36 (18.8%) patients had post-dose AEs (Table 2). The only post-dose AEs that occurred in ≥2% patients were schizophrenia (22 [11.5%]) and suicidal ideation (4 [2.1%]). *Weight increased in one placebo patient was the only AE identified using the post-dose AE definition of having a start date≥1 day after the last dose date among Withdrawal Population patients who had ≥2 follow-up days.

A total of 12 (6.5%) patients had post-dose AEs with a start date>1 day after the last dose date. There were no post-dose AEs with a start date>1 day after last dose date that occurred in ≥2% patients.

TABLE 2
Post-dose Adverse Events in Completed and Ongoing
Open-label Studies: Withdrawal Population

| | SEP-363856 25 to 100 mg/d | | | |
|---|---|---|---|---|
| | | | Post-dose AEs | |
| | | | with start date >1 | |
| System Organ Class/ | All Post-dose AEs | | day after last dose | |
| Preferred Term | (N = 192) | | (N = 186 ^{a}) | |
| Any AE | 36 | (18.8%) | 12 | (6.5%) |
| Infections and infestations | 1 | (0.5%) | 1 | (0.5%) |
| Influenza | 1 | (0.5%) | 1 | (0.5%) |
| Psychiatric disorders | 29 | (15.1%) | 7 | (3.8%) |
| Schizophrenia | 22 | (11.5%) | 3 | (1.6%) |
| Suicidal ideation | 4 | (2.1%) | 1 | (0.5%) |
| Psychotic disorder | 3 | (1.6%) | 2 | (1.1%) |
| Acute psychosis | 1 | (0.5%) | 1 | (0.5%) |
| Anxiety | 1 | (0.5%) | 0 | |
| Suicide attempt | 2 | (1.0%) | 1 | (0.5%) |
| Gastrointestinal disorders | 1 | (0.5%) | 1 | (0.5%) |
| Nausea | 1 | (0.5%) | 1 | (0.5%) |
| Vomiting | 1 | (0.5%) | 1 | (0.5%) |
| Hepatobiliary disorders | 1 | (0.5%) | 1 | (0.5%) |
| Hepatitis alcoholic | 1 | (0.5%) | 1 | (0.5%) |
| Musculoskeletal and | 1 | (0.5%) | 1 | (0.5%) |
| connective tissue disorders | | | | |
| Myalgia | 1 | (0.5%) | 1 | (0.5%) |
| Investigations | 6 | (3.1%) | 1 | (0.6%) |
| Weight decreased | 3 | (1.6%) | 0 | |
| Blood potassium decreased | 1 | (0.5%) | 1 | (0.5%) |
| Electrocardiogram QT prolonged | 1 | (0.5%) | 1 | (0.6%) |

| | | | |
|---|---|---|---|
| Heart rate increased | 1 | (0.5%) | 0 |

Abbreviations: d = day, mg = milligrams
^{a} Withdrawal Population subjects who had ≥2 follow-up days.
Note:
Withdrawal Population includes patients who took at least one dose of study medication and had at least one day of post-dose follow-up time. Rollover patients in double-blind studies, and ongoing subjects are excluded from the Withdrawal Population.
Note:
n is the number of patients with events. Percentages are based on the total number of patients in the treatment group in the Withdrawal Population. Patients are counted once per preferred term and system organ class.
Note:
Post-dose Adverse Events (AEs) are AEs with a start date after the date of last treatment exposure.

Post-dose AEs were also searched using a pre-defined list of preferred terms using the MedDRA version 22.0 SMQ: Drug withdrawal [20000102]. Preferred terms were tabulated using the MedDRA version used for each study (i.e., coding was not updated for studies that did not utilize MedDRA version 22.0). There were no withdrawal-related post-dose AEs as defined by the SMQ: Drug withdrawal identified in any of the completed Phase 2 studies or ongoing Phase 3 studies.

In conclusion, analysis of post-dose AEs as of the date of this evaluation did not reveal any signal for withdrawal effects.

### Example 2. A Double-Blind, Placebo-Controlled, Randomized Withdrawal Study to Evaluate SEP-363856 Physical Dependence in Adult Subjects with Schizophrenia

The primary objective of this study is to confirm the lack of physical dependence associated with SEP-363856, as indicated by the presence or absence of signs and/or symptoms of drug withdrawal upon abrupt SEP-363856 cessation (i.e., switch to placebo) in subjects with schizophrenia, compared to subjects with schizophrenia who receive continued SEP-363856 treatment. A secondary objective is to evaluate the safety and tolerability of SEP-363856 use in adults with schizophrenia. The overall study design, including the duration of the open-label treatment phase of 4-weeks, is consistent with published physical dependence studies (Stauffer 2014, Lerner 2015, Lerner 2019).

This is a double-blind, placebo-controlled, randomized withdrawal study comparing abrupt discontinuation of SEP-363856 (SEP-363856 switched to placebo) to continuous SEP-363856 treatment in male and female adult subjects with schizophrenia. The study consists of 4 periods: A Screening/Washout Period (up to 21 days), an Open-label Period (Days 1-35 SEP-363856), a double-blind, Randomized Withdrawal Period (Days 36-43) and a Follow-up Period (7 [+2] Days Post Last Dose). Study drug is taken at approximately the same time each evening at bedtime and is taken with or without food.

Approximately 40 subjects are enrolled in the Open-label Period in order to have at least 34 subjects randomized in the Randomized Withdrawal Period (17 in each group). Every attempt is made to enroll at least 30% of subjects from each sex. Subjects are outpatient for most of the Screening/Washout period with an option to have the subject in-clinic for up to 7 days prior to day 1, if deemed clinically necessary by the Investigator. Subjects are in-clinic or outpatient for the first 7 days of the Open-label period as subjects titrate up in dose, as deemed clinically necessary by the Investigator. Subjects are re-admitted on Day 29 (−3 days) and remain in-clinic through the end of the Randomized Withdrawal Period. Subjects are discharged at the end of the Randomized Withdrawal Period or remain in-clinic during part or for the entire Follow-up Period at the discretion of the Investigator.

Subjects are evaluated for eligibility during the Screening/Washout Period of up to 21 days, during which they are tapered off all neuropsychiatric medications (except permissible concomitant medications) in a manner consistent with labeling recommendations and conventional medical practices. Subjects are in-clinic for up to one week prior to Day 1, if deemed clinically necessary by the Investigator. Subjects are outpatient for most of the Screening/Washout period with the option to continue as an outpatient or in-clinic 1 week prior to entering the Open-label period, as deemed clinically necessary by the Investigator. Preferably, all neuropsychiatric medications are washed out within 3 days or 5 half-lives (whichever is longer) prior to the first dose of study drug.

Subjects who have successfully completed the washout of prior medication(s) and have met eligibility criteria enter the Open-label Period where they receive oral, open label SEP-363856 once daily (QD) over 35 days (Days 1 through 35) in order to establish a steady state baseline. Dosing of open-label SEP-363856 begins in the evening of Visit 2 (Day 1) and continues once-daily, in the evening at bedtime, for the remainder of the Open-label Period. During this 7-day titration period, subjects are in-clinic or be outpatient, per the discretion of the investigator. Subjects who are in-clinic are discharged on Day 8.

All subjects receive SEP-363856 at 50 mg/day for Days 1 through 3 then 75 mg/day for Days 4 through 7. Beginning on Day 8 through Day 35, subjects receive 100 mg/day for the remainder of the Open-label Period. Subjects unable to tolerate the 100 mg/day study medication are discontinued from the study and replaced. Subjects are outpatient for 3 weeks of this period (Day 8-28) and then are admitted to the clinical research unit (CRU) for the last week of the Open-label period (Day 29-35).

After the final dose in the Open-label Period on Day 35 (evening), subjects enter the 7-day double-blind Randomized Withdrawal Period and continue to be in-clinic. On Day 36 of the Randomized Withdrawal Period, subjects are randomized to continued SEP-363856 treatment or to placebo in a 1:1 ratio. Subjects are eligible for discharge beginning on Day 43 or early termination (ET) at the Investigator's discretion.

All subjects have safety Follow-up assessments 7 days (+2 days) following the last dose of study drug. Subjects who discontinue early from the study complete an early termination visit within 24 hours of last dose of study drug. Subjects are in-clinic from Day 1 through the end of the Randomized Withdrawal Period. Subjects are discharged at the end of the Randomized Withdrawal Period if they meet the discharge criteria, or they remain in-clinic during the Follow-up Period at the discretion of the Investigator.

To qualify for participation, subjects must meet the following main inclusion criteria:
Male or female subject between 18 and 65 years of age (inclusive).
Subject meets Diagnostic and Statistical Manual of Mental Disorders (DSM-5) criteria for a primary diagnosis of schizophrenia as established by clinical interview.
Subject must have a Clinical Global Impression-Severity Scale (CGI-S) score≤4 (normal to moderately ill).
Subject must have a Positive and negative symptoms scale (PANSS) total score≤80.
Subject must have a score of ≤4 on PANSS P7 (hostility) and G8 (uncooperativeness).
Subject has a body mass index (BMI) of at least 18.0 kg/m², but no more than 40.0 kg/m² at Screening.
Subject must have normal to mild symptoms on all individual items of the Simpson Angus Scale (SAS) (<2), Abnormal Involuntary Movement Scale (AIMS) (<3) and Barnes Akathisia Rating Scale (BARS) (<3) at Screening and Day 1.

The primary endpoint is the maximum change from the steady-state baseline (CSSBₘₐₓ) in the total score of the 20 item Physician Withdrawal Checklist (PWC-20) during the 7-day Randomized Withdrawal Period. The primary analysis is a non-inferiority test of the mean CSSBₘₐₓ in PWC-20 total score during the 7-day Randomized Withdrawal Period between the Placebo Group and the SEP-363856 Group in the Physical Dependency Analysis (“PDA”) population, assuming a non-inferiority margin of 4.71.

The following safety endpoints are also evaluated: overall incidence, frequency and severity of AEs; incidence of withdrawal-related AEs including, but may not be limited to, the Standardized Medical Dictionary for Regulatory Activities (MedDRA) Query: Drug withdrawal; vital signs (heart rate, blood pressure, respiratory rate, oral temperature); weight and body mass index (BMI); Columbia Suicide Severity Rating Scale (C-SSRS); and Abnormal Involuntary Movement Scale (AIMS), Barnes Akathisia Rating Scale (BARS), and Simpson-Angus Scale (SAS).

Other Endpoints to be evaluated include:
Occurrence of a clinically significant withdrawal syndrome during the 7-day Randomized Withdrawal Period defined as the CSSBₘₐₓ of PWC-20 total score reaching at least 4.71 points (i.e., increase ≥4.71)
Average change in PWC-20 total score from the steady-state baseline over the 7-day Randomized Withdrawal Period
Time to maximum PWC-20 total score during the 7-day Randomized Withdrawal Period
Change from Day 36 in PANSS total score and positive, negative and general psychopathology subscale scores during the 7-day Randomized Withdrawal Period
Change from Day 36 in CGI-S total score during the 7-day Randomized Withdrawal Period

Original terms used in the case report forms (CRFs) by Investigators or designees to identify AEs are coded using the Medical Dictionary for Regulatory Activities Terminology (MedDRA; Version 22.0 or higher). Overall summary of AEs includes all AEs for subjects while receiving SEP-363856 and up to 9 days after the last dose of SEP-363856. For Randomized Withdrawal Period, AEs starting on or after the first dose of study drug in the Randomized Withdrawal Period up to 9 days after the last dose of study drug (SEP-363856 or placebo) are summarized by randomized treatment group. AEs occurring before the open-label period are considered “pretreatment events” and are reported separately.

AEs are assigned to a treatment based on the time of occurrence in relation to the last treatment administered prior to the onset of the AE. AEs are summarized by treatment and by MedDRA SOC and PT. A listing of AEs, as well as a listing of deaths, SAEs, or TEAEs leading to discontinuation of study medication, are presented.

### REFERENCES CITED

Barnes T R. A rating scale for drug-induced akathisia. Br J Psychiatry. 1989; 154:672 676.
Cerovecki A, Musil R, Klimke A, Seemuller F, Haen E, Schennach R, et al. Withdrawal Symptoms and Rebound Syndromes Associated with Switching and Discontinuing Atypical Antipsychotics: Theoretical Background and Practical Recommendations. CNS Drugs (2013) 27:545-72.
Chouinard G, Samaha A N, Chouinard V A, Peretti C S, Kanahara N, Takase M, Iyo M. Antipsychotic-Induced Dopamine Supersensitivity Psychosis: Pharmacology, Criteria, and Therapy. Psychother Psychosom. 2017; 86(4):189-219.
Correll C U. From receptor pharmacology to improved outcomes: individualising the selection, dosing, and switching of antipsychotics. Eur Psychiatry (2010) 25:12-21. Food and Drug Administration (U.S.). Discussion Document for Patient-Focused Drug Development Public Workshop on Guidance 3: SELECT, DEVELOP OR MODIFY FIT-FOR-PURPOSE CLINICAL OUTCOME ASSESSMENTS (including Appendices) (Published at Patient-Focused Drug Development Guidance: Methods to Identify What is Important to Patients and Select, Develop or Modify Fit-for-Purpose Clinical Outcome Assessments, Meeting Oct. 15-16, 2018) (“FDA 2018”)
Food and Drug Administration (U.S.). Patient-Focused Drug Development: Methods to Identify What Is Important to Patients Guidance for Industry, Food and Drug Administration Staff, and Other Stakeholders (October 2019) (“FDA 2019”)
Food and Drug Administration (U.S.). Patient-Focused Drug Development: Collecting Comprehensive and Representative Input; Guidance for Industry, Food and Drug Administration Staff, and Other Stakeholders (June 2020) (“FDA 2020”).
Guy W. ECDEU Assessment Manual for Psychopharmacology—Revised (DHEW Publication No. ADM 76-338). Rockville, MD, US Department of Health, Education, and Welfare, 1976:218-22.
Horowitz M A, Jauhar S, Natesan S, Murray R M, Taylor D. A Method for Tapering Antipsychotic Treatment That May Minimize the Risk of Relapse. Schizophr Bull. 2021 Jul. 8; 47(4):1116-1129.
Lerner A and Klein M. Dependence, withdrawal and rebound of CNS drugs: an update and regulatory considerations for new drugs development. BRAIN COMMUNICATIONS 2019: 23 pages, Oxford University Press on behalf of the Guarantors of Brain.
Lerner A. Evaluation of Dependence and Withdrawal in Clinical Trials and Human Dependence Study-Design and Considerations. Session 2B: Regulatory Guidelines and Methodological Approaches to Assess Prescription Drug Abuse and Misuse During C NS Drug Development. 11th Annual International Society for CNS Clinical Trials and Methodology (ISCTM) Scientific Meeting Washington, DC Feb. 18, 2015. https://isctm.org/public_access/Feb2015/Presentations/Lerner.pdf
Montgomery S A, Asberg M. A new depression scale designed to be sensitive to change. Br J Psychiatry. 1979; 134:382-389.
Nilsson M E, Suryawanshi S, Gassmann-Mayer C, Dubrava S, McSorley P, Jiang K. Columbia Suicide Severity Rating Scale Scoring and Data Analysis Guide. February 2013. Available from http://cssrs.columbia.edu/wp-content/uploads/ScoringandDataAnalysisGuide-for-Clinical-Trials-1.pdf.
PCT Patent Publication No. WO 2020/118032
Rickels K, Garcia-Espana F, Mandos L A, Case G W. Physician Withdrawal Checklist (PWC-20). J Clin Psychopharmacol 2008; 28(4):447-451.
Simpson G N, Angus J W S. A rating scale for extrapyramidal side effects. Acta Psychiatr Scand. 1970; 212 (Suppl 44):S11-S19.
Stauffer V, Baygani S, Kinon B and Krikke-Workel J. A Short-Term, Multicenter, Placebo-Controlled, Randomized Withdrawal Study of a Metabotropic Glutamate 2/3 Receptor Agonist Using an Electronic Patient-Reported Outcome Device in Patients with Schizophrenia. J Clin Psychopharmacol 2014; 34: 552-558.
Tiihonen J, Tanskanen A, Taipale H. 20-Year Nationwide Follow-Up Study on Discontinuation of Antipsychotic Treatment in First-Episode Schizophrenia. Am J Psychiatry. 2018 Aug. 1; 175(8):765-773.
US 2020/0179336 A1 (Jun. 11, 2020)
US 2021/0315859 A1 (Oct. 14, 2021).
Viguera A C, Baldessarini R J, Hegarty J D, van Kammen D P, Tohen M. Clinical Risk Following Abrupt and Gradual Withdrawal of Maintenance Neuroleptic Treatment. Arch Gen Psychiatry. 1997; 54(1):49-55.
Williams J B. A structured interview guide for the Hamilton Depression Rating Scale. Arch Gen Psychiatry. 1988; 45:742-747.
Williams J B W. Structured interview guide for the Hamilton Anxiety Scale (SIGH-A). Biometrics Research Department, New York State Psychiatric Institute, New York, New York; Revision 15 Jan. 2008.

Throughout this application, various publications are referenced. The disclosures of these publications in their entireties are hereby incorporated by reference into this application in order to more fully describe the state of the art to which this disclosure pertains. It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of treating a neuropsychiatric disorder in a human subject in need thereof without significant physical dependency upon the cessation of treatment, comprising selecting and administering a therapeutically effective amount of ulotaront or a pharmaceutically acceptable salt thereof to the subject for an effective period until ceasing administration, wherein the subject does not develop physical dependency that is significantly greater than that caused by placebo-, optionally wherein the subject is at risk of developing, or has previously experienced, physical dependency.

2. A method of treating a neuropsychiatric disorder in a human subject in need thereof, comprising:
a. administering to the subject a therapeutically effective amount of ulotaront or a pharmaceutically acceptable salt thereof for an effective period, and
b. ceasing ulotaront administration after the effective period, wherein the subject does not develop significant physical dependency upon the cessation of treatment, optionally wherein the subject is at risk of developing, or has previously experienced, physical dependency.

3. (canceled)

4. A method of treating a neuropsychiatric disorder in a human subject in need thereof, comprising:
a. administering to the subject a therapeutically effective amount of ulotaront or a pharmaceutically acceptable salt thereof for an effective period, and
b. ceasing ulotaront administration after the effective period;
wherein the method occurs without emergence of one or more withdrawal-related adverse event(s), optionally wherein the subject does not develop significant physical dependency upon the cessation of treatment, optionally wherein the subject is at risk of developing, or the subject has previously experienced physical dependency.

5. The method of claim 4, wherein, upon the cessation of treatment, the subject does not develop physical dependency that is significantly greater than that caused by placebo.

6. The method of claim 4, wherein significant physical dependency is defined as the occurrence of a withdrawal-related adverse event comprising a clinically significant increase in the total score of the 20 item Physician Withdrawal Checklist (PWC-20); or wherein significant physical dependency is defined as the occurrence of a withdrawal-related adverse event comprising an increase in the total score of the 20 item Physician Withdrawal Checklist (PWC-20) of more than 4.71; or wherein significant physical dependency is defined as the occurrence of a withdrawal-related adverse event defined by Standardized Medical Dictionary for Regulatory Activities (MedDRA) Query: Drug withdrawal; or wherein significant physical dependency is defined as the occurrence of a withdrawal-related adverse event comprising one or more symptom(s) of neuropsychiatric medication withdrawal selected from somatic symptoms, mood symptoms, cognitive symptoms, fatigue symptoms, and gastrointestinal symptoms; or wherein significant physical dependency is defined as the occurrence of a withdrawal-related adverse event selected from cholinergic withdrawal symptoms, dopaminergic withdrawal symptoms (nigrostriatal), serotonin withdrawal symptoms, histaminergic withdrawal symptoms, dopaminergic withdrawal symptoms (mesolimbic or striatal), and adrenergic withdrawal symptoms.

7-10. (canceled)

11. The method of claim 4, wherein the effective period is a term≥2 weeks, ≥4 weeks, ≥8 weeks, ≥12 weeks, ≥26 weeks, ≥one year, or ≥two years; or wherein the effective period is sufficient for a clinically significant improvement from the neuropsychiatric disorder; or wherein the effective period is sufficient for full recovery from the neuropsychiatric disorder; or wherein the effective period is sufficient for full recovery from the neuropsychiatric disorder, as evidenced by a lack of diagnosis of the neuropsychiatric disorder under criteria set forth in DSM-5; or wherein the effective period is sufficient for full recovery from the neuropsychiatric disorder for a term≥3 months, ≥6 months, 1 year, 18 months, or ≥2 years; or wherein the effective period is sufficient to judge the ineffectiveness of ulotaront against the subject's neuropsychiatric disorder; or wherein the effective period is sufficient to judge the ineffectiveness of ulotaront against the subject's neuropsychiatric disorder, as evidenced by the failure of a clinically significant improvement from the neuropsychiatric disorder; or wherein the effective period is sufficient to judge the ineffectiveness of ulotaront against the subject's neuropsychiatric disorder, as evidenced by a continued diagnosis of the neuropsychiatric disorder under criteria set forth in DSM-5.

12-18. (canceled)

19. The method of claim 4, wherein the effective period is sufficient to judge the ineffectiveness of ulotaront against the subject's neuropsychiatric disorder, as evidenced by one or more of:
a. inadequate clinical response for acute symptoms despite dose optimization and adequate duration of treatment trial;
b. poor control of chronic symptoms and persistence of functional disabilities during maintenance therapy;
c. relapse despite adequate prophylactic or maintenance treatment of the neuropsychiatric disorder; and
d. persistence of certain symptoms of the neuropsychiatric despite adequate doses of ulotaront.

20. The method of claim 4, wherein the ceasing is abrupt.

21. The method of a claim 4, wherein the ceasing is tapered.

22. The method of claim 4, wherein the subject is at risk of one or more symptom(s) or syndrome(s) of neuropsychiatric medication withdrawal.

23. The method of claim 4, wherein the subject has previously experienced one or more symptom(s) or syndrome(s) of neuropsychiatric medication withdrawal.

24. The method of claim 4, wherein the method occurs without emergence of one or more symptom(s) or syndrome(s) of neuropsychiatric medication withdrawal.

25. The method of claim 4, wherein the subject is at risk of, or the subject has previously experienced, or the method occurs without emergence of, a withdrawal-related adverse event comprising a clinically significant increase in the total score of the 20 item Physician Withdrawal Checklist (PWC-20); or a withdrawal-related adverse event comprising an increase in the total score of the 20 item Physician Withdrawal Checklist (PWC-20) of more than 4.71; or a withdrawal-related adverse event defined by Standardized Medical Dictionary for Regulatory Activities (MedDRA) Query: Drug withdrawal.

26. (canceled)

27. (canceled)

28. The method of claim 4, wherein the subject is at risk of, or the subject has previously experienced, or the method occurs without emergence of, a withdrawal-related adverse event comprising one or more symptom(s) of neuropsychiatric medication withdrawal selected from somatic symptoms, mood symptoms, cognitive symptoms, fatigue symptoms, and gastrointestinal symptoms.

29. The method of claim 4, wherein the subject is at risk of, or the subject has previously experienced, or the method occurs without emergence of, a withdrawal-related adverse event comprising one or more somatic symptom(s) of neuropsychiatric medication withdrawal selected from (i) insomnia, (ii) diaphoresis, (iii) tremor-tremulousness, (iv) headaches, and (v) muscle aches or stiffness; or a withdrawal-related adverse event comprising one or more mood symptom(s) of neuropsychiatric medication withdrawal selected from (i) anxiety-nervousness, (ii) irritability, (iii) dysphoric mood-depression, (iv) restlessness-agitation, and (v) difficulty concentrating, remembering; or a withdrawal-related adverse event comprising one or more cognitive symptom(s) of neuropsychiatric medication withdrawal selected from (i) poor coordination, (ii) dizziness-lightheadedness, (iii) increased acuity for sound, smell, touch, and (iv) depersonalization-derealization; or a withdrawal-related adverse event comprising one or more fatigue symptom(s) of neuropsychiatric medication withdrawal selected from (i) fatigue-lethargy-lack of energy, (ii) weakness, and (iii) paresthesias; or a withdrawal-related adverse event comprising one or more gastrointestinal symptom(s) of neuropsychiatric medication withdrawal selected from (i) loss of appetite, (ii) nausea-vomiting, and (iii) diarrhea.

30-33. (canceled)

34. The method of claim 4, wherein the subject is at risk of, or the subject has previously experienced, or the method occurs without emergence of, a withdrawal-related adverse event comprising one or more withdrawal symptom(s) of mild, moderate, or severe magnitude.

35. The method of claim 4, wherein the subject is at risk of, or the subject has previously experienced, or the method occurs without emergence of, a withdrawal-related adverse event comprising one or more syndrome(s) of neuropsychiatric medication withdrawal selected from:
a. cholinergic syndrome evidenced by one or more of nausea, vomiting, headache, restlessness, anxiety, insomnia, fatigue, malaise, myalgia, diaphoresis, rhinitis, paresthesia, and loose bowels;
b. dopaminergic syndrome evidenced by one or more of withdrawal dyskinesia, akathisia, dystonia, tardive dyskinesia; and
c. rebound psychosis evidenced by one or more of psychosis above pre-treatment levels, illusions, hallucinations, and catatonia.

36. The method of claim 4, wherein the subject is at risk of, or the subject has previously experienced, or the method occurs without emergence of, a withdrawal-related adverse event selected from cholinergic withdrawal symptoms, dopaminergic withdrawal symptoms (nigrostriatal), serotonin withdrawal symptoms, histaminergic withdrawal symptoms, dopaminergic withdrawal symptoms (mesolimbic or striatal), and adrenergic withdrawal symptoms.

37. The method of claim 4, wherein the subject is at risk of, or the subject has previously experienced, or the method occurs without emergence of, a withdrawal-related adverse event comprising cholinergic withdrawal symptoms selected from agitation, insomnia, anxiety or depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion or disorientation, hypothermia, and sweating; or a withdrawal-related adverse event comprising dopaminergic withdrawal symptoms (nigrostriatal) selected from withdrawal dyskinesia, parkinsonism, neuroleptic malignant syndrome, and akathisia; or a withdrawal-related adverse event comprising serotonin withdrawal symptoms selected from flu-like symptoms, sweating or chills, dizziness, light-headedness or tachycardia, paresthesia, electric chock sensations, anxiety, agitation, low mood, insomnia, nightmares, nausea, vomiting, diarrhea, confusion, and decreased concentration; or a withdrawal-related adverse event comprising histaminergic withdrawal symptoms selected from irritability, insomnia, agitation, depressed affect, loss of appetite or nausea, tremulousness, incoordination, and lethargy or amnesia; or a withdrawal-related adverse event comprising dopaminergic withdrawal symptoms (mesolimbic or striatal) selected from auditory hallucinations, persecutory delusions, and other psychotic symptoms; or a withdrawal-related adverse event comprising adrenergic withdrawal symptoms selected from headache, anxiety or agitation, hypertension, tachycardia, angina, palpitations, risk of myocardial infarction, pre-syncope, tremulousness, and sweating.

38-46. (canceled)

47. The method of claim 4 comprising, after the effective period, ceasing ulotaront administration for the neuropsychiatric disorder, for a period≥3 months, ≥6 months, ≥1 year, ≥18 months, or ≥2 years.

48. The method of claim 4, wherein the therapeutically effective amount is 25-150 mg/day or 25-100 mg/day or 50-125 mg/day or 50-100 mg/day administered orally; or wherein the therapeutically effective amount is 25 mg/day, 50 mg/day, 75 mg/day, 100 mg/day, 125 mg/day, or 150 mg/day, orally administered.

49. (canceled)

50. The method of claim 4, wherein the therapeutically effective amount is administered once daily in the fed or fasted state.

51. The method of claim 4, wherein the ulotaront is administered as the hydrochloride salt.

52. The method of claim 4, wherein the neuropsychiatric disorder is selected from schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, aggression, delirium, Parkinson's psychosis, excitative psychosis, Tourette's syndrome, organic or NOS psychosis, seizure, agitation, post-traumatic stress disorder, behavior disorder, neurodegenerative disease, Alzheimer's disease, Parkinson's disease, dyskinesias, Huntington's disease, dementia, mood disorder, anxiety, affective disorders (e.g., depression, e.g., major depressive disorder and dysthymia; bipolar disorder, e.g., bipolar depressive disorder; manic disorder; seasonal affective disorder; and attention deficit disorder (ADD) and attention deficit hyperactivity disorder (ADHD)), obsessive-compulsive disorder, vertigo, epilepsy, pain (e.g., neuropathic pain, sensitization accompanying neuropathic pain, and inflammatory pain), fibromyalgia, migraine, cognitive impairment, movement disorder, restless leg syndrome (RLS), multiple sclerosis, sleep disorder, sleep apnea, narcolepsy, excessive daytime sleepiness, jet lag, drowsy side effect of medications, insomnia, substance abuse or dependency (e.g., nicotine, cocaine), addiction, eating disorder, sexual dysfunction, hypertension, emesis, Lesche-Nyhane disease, Wilson's disease, autism, Huntington's chorea, and premenstrual dysphoria.

53-56. (canceled)
